(19) United States
(12) Patent Application Publication
(10) Pub. No.: US 2024/0261780 A1
(43) Pub. Date: Aug. 8, 2024

(54) SAMPLE COLLECTION DEVICE AND SYSTEM

(71) Applicant: 3M INNOVATIVE PROPERTIES COMPANY, St. Paul, MN (US)

(72) Inventors: Michael R. Berrigan, Oakdale, MN (US); Alan R. Dombrowski, Woodbury, MN (US); Laura R. Nereng, Woodbury, MN (US); Audrey A. Sherman, Woodbury, MN (US); Brett J. Sitter, Marine on St. Croix, MN (US); Narina Y. Stepanova, Woodbury, MN (US); Michael C. Wohl, Bethesda, MD (US)

(21) Appl. No.: 18/564,500

(22) PCT Filed: May 19, 2022
(86) PCT No.: PCT/IB2022/054683

§ 371 Date: Nov. 27, 2023

Related U.S. Application Data
(60) Provisional application No. 63/202,140, filed on May 28, 2021.
(60) Provisional application No. 63/227,534, filed on Jul. 30, 2021.

(51) Int. Cl. B01L 3/00

(52) U.S. Cl. CPC B01L 3/5023; B01L 3/502715; B01L 3/502753; B01L 2300/0825; B01L 2400/0406; B01L 2400/0683

(57) ABSTRACT

A sample collection system includes a housing comprising a first part and a second part, the first and second parts being movable relative to one another; porous sample collection media disposed along an airflow path in the first part; and an assay disposed on the second part and constructed to receive a sample captured by the porous sample collection media. The first and second parts may be slidably movable relative to one another. The first and second parts may be rotatably movable relative to one another. Moving the first and second parts relative to one another aligns the porous sample collection media with at least a portion of the assay. The assay may be a lateral flow assay or a vertical flow assay. The media is constructed to collect a sample from exhalation airflow. A collected sample may be eluted from the media onto the assay using a liquid.

### FIELD

The present disclosure relates to a sample collection device and system. The present disclosure relates to a bioaerosol collection device and system.

### BACKGROUND

Diagnostic tests used to test for the presence of a virus or other pathogen in the airways, throat, or nasopharynx typically involve the insertion of a swab into the back of the nasal passage, the mid-turbinate area of the nasal passage, the anterior nares, or the throat to obtain a sample. The swab is then inserted into a container and analyzed or sent to a lab for processing. Other diagnostic tests involve collecting a saliva sample and then placing it in a container. Currently available at-home viral tests (e.g., COVID-19 tests) involve a nasal swab and a test kit (for example, the Ellume™ test, the Abbot™ BinaxNOW™ test, and the Lucira™ All-in-One test kit). Tests that utilize nasal swab samples or saliva contend with contaminants that can interfere with the various diagnostic tests. As a result, these sample types require a purification step when using RT-PCR molecular testing.

### SUMMARY

There is a need for an inexpensive, simple to use, and reliable sample collection system that may be used by laypeople for testing for the presence of a target virus, target pathogen, or other target analyte, in a collected sample. The sample collection system may include a sample collection device for collecting a sample from exhalation airflow and a testing assay to determine the presence or absence of virus or other pathogen in the collected sample.

It is desirable to provide a system that includes both a sample collector device and rapid antigen testing in an integrated system. The integrated system may advantageously be self-contained and optionally sterile. A self-contained and sterile system may improve accuracy and reliability of pathogen testing due to the reduced contamination and background noise, unlike swabs and other test collection devices which may be contaminated upon use and/or during testing.

It is further desirable to provide a system which, after sample collection and optional testing, remains closed and self-contained to contain any potential virus or pathogen, and which may be safely disposed of among ordinary waste collection.

According to an embodiment, a sample collection system includes a housing comprising a first part and a second part, the first and second parts being movable relative to one another, porous sample collection media disposed along an airflow path in the first part; and an assay disposed on the second part and constructed to receive a sample captured by the porous sample collection media. The airflow path defines a through opening in the first part. The first part may include a screen disposed in the airflow path in front of the porous sample collection media.

The first and second parts may be slidably movable relative to one another. The first and second parts may be rotatably movable relative to one another. The first and second parts may be connected to one another by a hinge. According to an embodiment, moving the first and second parts relative to one another aligns the porous sample collection media with at least a portion of the assay.

The housing may further include a liquid inlet constructed to receive a liquid and to direct the liquid onto the porous sample collection media. The housing may include a liquid reservoir containing a metered dose of liquid. The liquid reservoir may be pierceable, frangible, or rupturable. The liquid reservoir may be disposed on a third part of the housing.

The porous sample collection media is constructed to capture a sample of viruses, pathogens, or other analytes from exhalation airflow. The porous sample collection media may be made of nonwoven material. The nonwoven material may include polylactic acid, polypropylene, or a combination thereof. The nonwoven material may carry an electrostatic charge. The assay may be a lateral flow assay or a vertical flow assay. The assay is constructed to determine the presence or absence of a target virus, pathogen, or analyte in the collected sample.

According to an embodiment, a kit includes the sample collection system and instructions for collecting a sample and testing the sample using the assay. The instructions may include instructions to: exhale along the airflow path to capture a sample in the porous sample collection media; move the first and second parts relative to one another to align the porous sample collection media with the assay; apply a liquid to the porous sample collection media; and read a result in a result display of the assay.

### BRIEF DESCRIPTION OF FIGURES

FIGS. 1A-1C are perspective views of a sample collection system according to an embodiment.

FIG. 2 is a perspective view of a first part of the sample collection system of FIG. 1A.

FIG. 3A is a perspective view of a second part of the sample collection system of FIG. 1A.

FIG. 3B is a cross-sectional view of a second part of the sample collection system of FIG. 1A.

FIG. 4A is a partial cross-sectional perspective view of the sample collection system of FIG. 1A.

FIG. 4B is a cross-sectional perspective view of the sample collection system of FIG. 1A.

FIGS. 5A-5C are perspective views of a sample collection system according to an embodiment.

FIG. 6 is a perspective view of a first part of the sample collection system of FIG. 5A.

FIG. 7 is a cross-sectional perspective view of the sample collection system of FIG. 5A in use.

FIG. 8 is a cross-sectional perspective view of the first part of the sample collection system of FIG. 5A.

FIGS. 9A and 9B are perspective views of a sample collection system in a first position according to an embodiment.

FIG. 9C is a perspective view of the sample collection system of FIG. 9A in a second position.

FIG. 10A is a perspective view of a sample collection system in a first position according to an embodiment.

FIG. 10B is a perspective view of the sample collection system of FIG. 10A in a partially closed position.

FIG. 10C is a perspective view of the sample collection system of FIG. 10A in a second position.

### DEFINITIONS

All scientific and technical terms used herein have meanings commonly used in the art unless otherwise specified. The definitions provided herein are to facilitate understanding of certain terms used frequently herein and are not meant to limit the scope of the present disclosure.

Unless otherwise indicated, the terms “polymer” and “polymeric material” include, but are not limited to, organic homopolymers, copolymers, such as for example, block, graft, random and alternating copolymers, terpolymers, etc., and blends and modifications thereof. Furthermore, unless otherwise specifically limited, the term “polymer” shall include all possible geometrical configurations of the material. These configurations include, but are not limited to, isotactic, syndiotactic, and atactic symmetries.

The terms “downstream” and “upstream” refer to a relative position based on a direction of exhalation airflow through the device. For example, the upstream-most element of the device is the mouthpiece element, and the downstream-most element of the device is the exhalation outlet element.

All headings provided herein are for the convenience of the reader and should not be used to limit the meaning of any text that follows the heading, unless so specified.

The term “i.e.” is used here as an abbreviation for the Latin phrase id est, and means “that is,” while “e.g.” is used as an abbreviation for the Latin phrase exempli gratia and means “for example.”

The term “not substantially” as used here has the same meaning as “not significantly,” and can be understood to have the inverse meaning of “substantially,” i.e., modifying the term that follows by not more than 25%, not more than 10%, not more than 5%, or not more than 2%.

All scientific and technical terms used herein have meanings commonly used in the art unless otherwise specified. The definitions provided herein are to facilitate understanding of certain terms used frequently herein and are not meant to limit the scope of the present disclosure.

The term “about” is used here in conjunction with numeric values to include normal variations in measurements as expected by persons skilled in the art and is understood have the same meaning as “approximately” and to cover a typical margin of error, such as ±5% of the stated value. Moreover, unless otherwise indicated, all numbers expressing quantities, and all terms expressing direction/orientation (e.g., vertical, horizontal, parallel, perpendicular, etc.) in the specification and claims are to be understood as being modified in all instances by the term “about.”

Terms such as “a,” “an,” and “the” are not intended to refer to only a singular entity but include the general class of which a specific example may be used for illustration.

The terms “a,” “an,” and “the” are used interchangeably with the term “at least one.” The phrases “at least one of” and “comprises at least one of” followed by a list refers to any one of the items in the list and any combination of two or more items in the list.

As used here, the term “or” is generally employed in its usual sense including “and/or” unless the content clearly dictates otherwise. The term “and/or” means one or all of the listed elements or a combination of any two or more of the listed elements.

The recitations of numerical ranges by endpoints include all numbers subsumed within that range (e.g., 1 to 5 includes 1, 1.5, 2, 2.75, 3, 3.80, 4, 5, etc. or 10 or less includes 10, 9.4, 7.6, 5, 4.3, 2.9, 1.62, 0.3, etc.). Where a range of values is “up to” or “at least” a particular value, that value is included within the range.

As used here, “have”, “having”, “include”, “including”, “comprise”, “comprising” or the like are used in their open-ended sense, and generally mean “including, but not limited to.” It will be understood that “consisting essentially of,” “consisting of,” and the like are subsumed in “comprising” and the like. As used herein, “consisting essentially of,” as it relates to a composition, product, method or the like, means that the components of the composition, product, method or the like are limited to the enumerated components and any other components that do not materially affect the basic and novel characteristic(s) of the composition, product, method or the like.

The term “substantially” as used here has the same meaning as “significantly,” and can be understood to modify the term that follows by at least about 90%, at least about 95%, or at least about 98%. The term “not substantially” as used here has the same meaning as “not significantly,” and can be understood to have the inverse meaning of “substantially,” i.e., modifying the term that follows by not more than 10%, not more than 5%, or not more than 2%.

The words “preferred” and “preferably” refer to embodiments that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

Any direction referred to here, such as “front,” “back,” “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

Any direction referred to here, such as “top,” “bottom,” “left,” “right,” “upper,” “lower,” and other directions and orientations are described herein for clarity in reference to the figures and are not to be limiting of an actual device or system or use of the device or system. Devices or systems as described herein may be used in a number of directions and orientations.

### DETAILED DESCRIPTION

The present disclosure relates to a sample collection device and system. The present disclosure relates to a bioaerosol sample collection device. The present disclosure further relates to a system that includes both sample collection and testing capabilities.

The sample collection system includes a sample collection device with porous sample collection media along an airflow path defined by the device housing. The porous sample collection media is constructed to capture viruses, pathogens, or other analytes, carried in an exhalation airflow. The system may further include a sample testing assay. A liquid may be passed through the porous sample collection media to elute the sample, including any viruses, pathogens, or other analytes, bound to the porous sample collection media, forming an eluent. The eluent may then be analyzed using the assay. The eluent may be directed from the sample collection media to the assay. The liquid may be provided as a metered dose of liquid housed in a liquid reservoir. The liquid may alternatively be applied from a separate applicator. The device or container including the liquid could have a tamper-indicating portion that indicates if the liquid has been tampered with so that the user could see that before use. An exemplary tamper-indicating device or container is one that has a foil or similar cover. If the cover has been pierced or removed, that would be evident to the user. Other tamper-indicating features include the delivery of the liquid resulting in breakage or damage to the liquid container such that the user could see that it had been used or tampered with, colored dye indicating user or tampering, or breakpoints (e.g. perforations, thin walls, torque breaking plastic portions, and/or spring like designs) that indicate to the user if the liquid containing device has been used or tampered with.

According to an embodiment, the sample collection device includes a housing. The housing may include a first part and a second part. The first and second parts are movable relative to one another. The porous sample collection media may disposed along an airflow path in the first part. The assay may be disposed on (e.g., attached to) the second part. The porous sample collection media is constructed to capture a sample from an exhalation of a user. The assay is constructed to receive the sample captured by the porous sample collection media.

In some embodiments, the assay may be a separate element from the sample collection device. The assay may be configured to attach to (e.g., slide or snap in) the sample collection device. The sample collection device may include a receptable for receiving the assay. The assay may be a replacement element with the sample collection device. The assay may be integral with the sample collection device. The assay may form a unitary element with the housing of the sample collection device.

The housing may define one or more openings that form the airflow path. In some cases, the housing defines only a single opening that forms the airflow path. The single opening may be a through hole in a structure (e.g., the first part) of the housing. According to an embodiment, the housing includes a first part and a second part, and a through opening in the first part defining the airflow path. The porous sample collection media may be disposed or fixed along the airflow path. For example, the porous sample collection media may be fixed or attached to the housing such that the porous sample collection media covers the opening (or at least one of the openings) forming the airflow path.

The housing may include a liquid inlet constructed to receive a liquid for eluting the sample. The liquid inlet may be constructed to direct the liquid onto the porous sample collection media. In some embodiments, the housing includes a liquid reservoir containing a metered dose of liquid. The liquid reservoir may be provided on a third part of the housing. The third part may be movable relative to the first part, the second part, or both the first and second parts of the housing. Alternatively, the liquid may be provided separately, e.g., in a dropper or other applicator. The liquid reservoir containing the metered dose of liquid may be deformable and configured to discharge fluid from the metered fluid dose element upon application of pressure to the liquid reservoir. In some embodiments the liquid reservoir is pierceable, frangible, or rupturable.

According to an embodiment, the first and second parts may be slidably movable relative to one another. For example, one of the first and second parts may include a track or pocket constructed to receive the other part. The first and second parts may be rotatably movable relative to one another. The first and second parts may be rotatably movable about a hinge connecting the first and second parts. According to an embodiment, moving the first and second parts relative to one another aligns (e.g., registers) the porous sample collection media with at least a portion of the assay. For example, moving the first and second parts relative to one another may align the porous sample collection media with a sample receiving area of the assay. Moving the first and second parts relative to one another may cause the porous sample collection media to come into contact with (e.g., touch) the assay. Moving the first and second parts relative to one another may cause the porous sample collection media to come into contact with (e.g., touch) a sample receiving area of the assay.

In embodiments that include a third part of the housing that provides a liquid reservoir, the third part may be rotatably movable relative to the first part, the second part, or both the first and second parts of the housing. The third part may be movable about a second hinge. For example, the first part and the third part may be disposed parallel to one another at opposing sides of the second part, each connected to the second part by a hinge. In this configuration, the housing is constructed for the first part to be moved first against the second part and the third part then to be moved against the first part, overlapping the aligned porous sample collection media and assay. Overlapping the third part with the first part and second part may also cause the liquid reservoir to rupture, break, or be pierced, thus releasing the liquid from the liquid reservoir.

The liquid dispensed onto the porous sample collection media may be an aqueous liquid. The liquid may be a buffer solution. The liquid may be an aqueous buffer solution. The liquid may be a saline solution. The liquid may include a surfactant. The liquid may have a contact angle of greater than 90 degrees when measured on the porous sample collection media. The liquid may be a saline solution including a surfactant. The liquid (e.g., a buffer or a saline solution) may include from 0.1 wt-% or more or 0.5 wt-% or more, and up to 1 wt-% or up to 2 wt-% of surfactant. When provided as a metered dose, the liquid may have a volume of 50 μL to 500 μL.

The liquid may be applied through the fluid inlet port and applied onto the loaded porous sample collection media. The liquid may travel through the surface and thickness of the loaded porous sample collection media and flow off of the porous sample collection media carrying any virus, pathogen, or other analyte, that was present on the loaded porous sample collection media. This loaded liquid may then be collected and tested, as described herein.

The housing may include a pressure element (e.g., a protrusion or a spring) constructed to apply pressure to the porous sample collection media to press the porous sample collection media against the assay. The pressure element may be constructed to cause more eluent to flow from the porous sample collection media to the assay.

The user may exhale into the sample collection system and load the porous sample collection media with a sample of the exhalation airflow to form a loaded porous sample collection media. For example, the user may exhale through the single opening or through the air inlet. The housing may be constructed such that by exhaling through the single opening or through the air inlet, the exhalation airflow passes through the porous sample collection media. The porous sample collection media is constructed to capture viruses, other pathogens, or other analytes, from the exhalation airflow. The user may then move the first and second parts of the housing relative to one another to bring the loaded porous sample collection media and the assay together. For example, moving the first and second parts may align the porous sample collection media and the assay, or may cause the porous sample collection media and the assay to touch one another. The user may then apply a liquid to the loaded porous sample collection media to elute the captured sample onto the assay. The user may apply the liquid by using a liquid reservoir containing a metered dose of liquid. The user may test the eluent for the presence of a virus, pathogen, or other analyte using the assay. The testing may take place with the loaded porous sample collection media in place in the sample collection system.

According to an embodiment, the sample collection system may be closed before use, after use, or both before and after use to provide an integrated self-contained unit. The terms “closed” and “closing” are used here to refer to moving the first and second parts to a position where the porous sample media is not accessible for use and/or the airflow path is closed. For example, in embodiments where the first and second parts are connected by a hinge, the first and second parts may be closed like a book. In embodiments where the parts are slidably movable, the first and second parts may be closed by sliding one part all the way, as far as possible, inside the other. Even in embodiments where the sample collection system is constructed from two or more separate parts, the parts of the system may be coupled together to form a singular self-contained unit. For example, the system may be provided as a flat self-contained unit that can be easily fit inside an envelope or other small shipping container or display unit for shipment, delivery, or sale to a user. The self-contained unit may have a compact size and may be conveniently carried in a pocket or purse. The closed self-contained unit may be safely disposed of after use among ordinary waste disposal. The housing may further include a locking mechanism that is constructed to lock when the first and second parts are moved to align the porous sample collection media with the assay. The locking mechanism may prevent opening the first and second parts after the porous sample collection media and the assay have been aligned. The locking mechanism may prevent opening the first and second parts after use of the assay.

The assay included in the sample collection system may be any suitable assay. In some embodiments, the assay is a lateral flow assay (“LFA”) or a vertical flow assay (“VFA”). LFAs and VFAs are generally paper-based platforms for the detection and quantification of analytes in complex mixtures, including biological samples such as saliva, urine, etc. LFAs and VFAs are typically easy to use and can be used both by professionals in a health care setting or laboratory as well as by lay persons at home. Typically, a liquid sample is placed on the assay in a sample receiving region and is wicked by capillary flow along the device to a test region. LFAs and VFAs are typically based on antigens or antibodies that are immobilized in the test region and that selectively react with the analyte of interest. The result is typically displayed within 5 to 30 minutes. LFAs and VFAs can be tailored for the testing of a variety of viruses and other pathogens, as well as many other types of analytes. According to an embodiment, the assay used in the sample collection system of the present disclosure is constructed for the detection of a target virus, target pathogen, or other target analyte. According to an embodiment, the assay used in the sample collection system of the present disclosure is constructed for the detection of a target virus, target pathogen, or other target analyte, that may be present in the exhalation air flow of a subject.

In some embodiments, the porous sample collection media is a nonwoven material. In some embodiments, the nonwoven material is porous. In some embodiments, the nonwoven material has an electrostatic charge. The electrostatic charge may enable capturing pathogens, viruses, or other analytes from an exhalation airflow.

In some cases, the porous sample collection media may be a hydrophobic nonwoven material. In other cases, the porous sample collection media may be a hydrophilic nonwoven material. The porous sample collection media may be a hydrophobic nonwoven material carrying an electrostatic charge configured to capture pathogens, viruses, or other analytes from an exhalation airflow. The porous sample collection media may be a hydrophilic nonwoven material carrying an electrostatic charge configured to capture pathogens, viruses, or other analytes from an exhalation airflow. The term “hydrophobic” refers to a material having a water contact angle of 90 degrees or greater, or from about 90 degrees to about 170 degrees, or from about 100 degrees to about 150 degrees. The term “hydrophilic” refers to a material having a water contact angle of less than 90 degrees. Water contact angle is measured using ASTM D5727-1997 Standard test method for surface wettability and absorbency of sheeted material using an automated contact angle tester.

The porous sample collection media may be formed of any suitable material that is capable of capturing viruses, pathogens, or other analytes from exhalation airflow and releasing the captured viruses, pathogens, or other analytes upon being contacted with an eluent, such as a saline solution. The porous sample collection media may be formed of polymeric material. The porous sample collection media may be formed of a polyolefin. Examples of suitable polyolefins include polypropylene, polylactic acid, and the like, and a combination thereof. In one embodiment the porous sample collection media is formed of polypropylene. In one embodiment the porous sample collection media is formed of polylactic acid. One illustrative porous sample collection media is commercially available from 3M Company (St. Paul MN, U.S.A.) under the trade designation FILTRETE Smart MPR 1900 Premium Allergen, Bacteria & Virus Air Filter Merv 13.

While the porous sample collection media is illustrated here as defining a planar element, it is understood that the porous sample collection media may define any shape when disposed within the housing and along the airflow path. For example, the sample collection media may be pleated. In some embodiments, the pleat frequency is between about 1 pleat per 0.6 cm of media and about 1 pleat per 2 mm of media. In some embodiments, the pleat height is between about 2 mm and about 4 mm.

The porous sample collection media may have a thickness (orthogonal to the major plane) of 200 μm or greater or 250 μm or greater. The porous sample collection media may have a thickness of 750 μm or less or 1000 μm or less. The porous sample collection media may have a thickness of in a range from 200 to 1000 μm, or from 250 to 750 μm. The porous sample collection media may have major plane surface area (of one side) of 1 cm2 or greater or 2 cm2 or greater. The porous sample collection media may have major plane surface area of 3 cm2 or less or 4 cm2 or less. The porous sample collection media may have major plane surface area in a range from 1 cm2 to 4 cm2, or 2 cm2 to 3 cm2.

The housing may be formed of a rigid material, such as plastic or a paper-based material such as cardboard or cardstock. In some embodiments, the housing is made of plastic. In some embodiments, at least a portion of the housing is transparent. For example, the housing may include transparent material in an area of a result display of the assay. The housing may include a viewing window (either transparent material or an opening) in the area of the result display. In some cases, the entire housing may be made of a transparent material.

The housing may include a pre-filter or screen disposed in the airflow path in front (upstream) of the porous sample collection media. For example, the first part of the housing may include a screen disposed in the airflow path in front of the porous sample collection media. The screen may be constructed to catch larger particles (larger than viruses or pathogens) and prevent such particles from reaching the porous sample collection media. The exhalation airflow passes through a thickness of the pre-filter or screen. The pre-filter or screen at least partially occludes the air flow path. In some cases, the pre-filter or screen may have a major plane that is orthogonal to the direction of the exhalation airflow passing through the thickness of the pre-filter or screen. The pre-filter or screen may be a non-woven layer configured to filter out larger particles from the exhalation airflow passing through the pre-filter or screen. In some cases, the pre-filter or screen may be a non-woven layer that does not have an electrostatic charge. In some embodiments, the pre-filter or screen does not capture significant amounts of viral material, pathogen material, or other analyte material, and instead allows them to transmit through the pre-filter or screen. In some embodiments, the pre-filter or screen is made of or includes at least one of a plastic mesh, a woven net, a needle-tacked fibrous web, a knitted mesh, an extruded net, and/or a carded or spunbond coverstock.

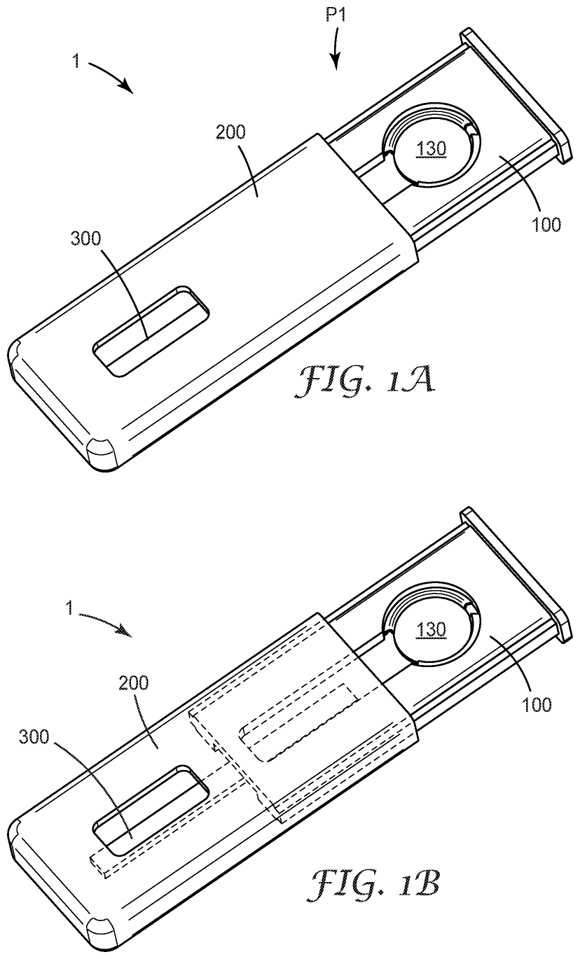

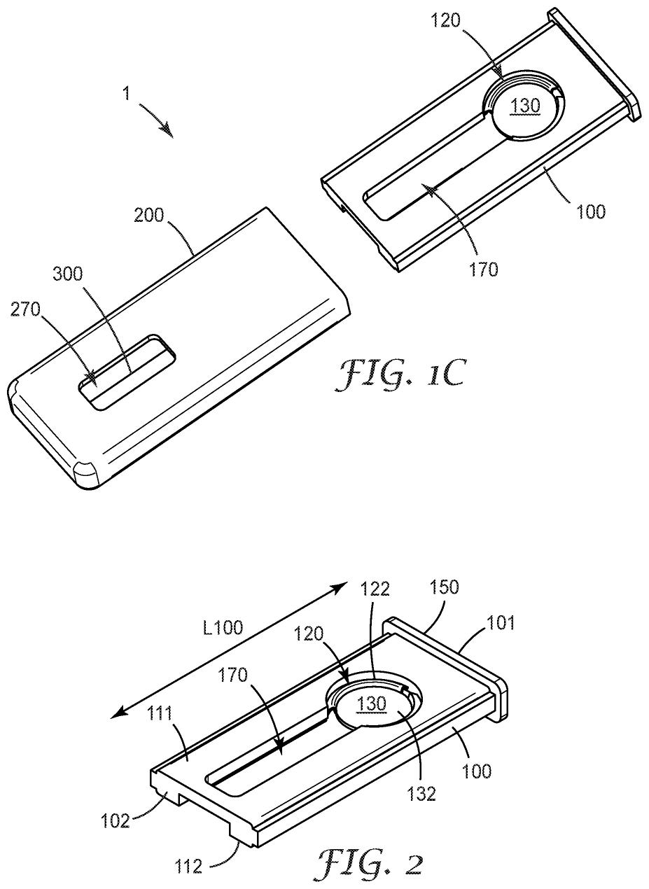

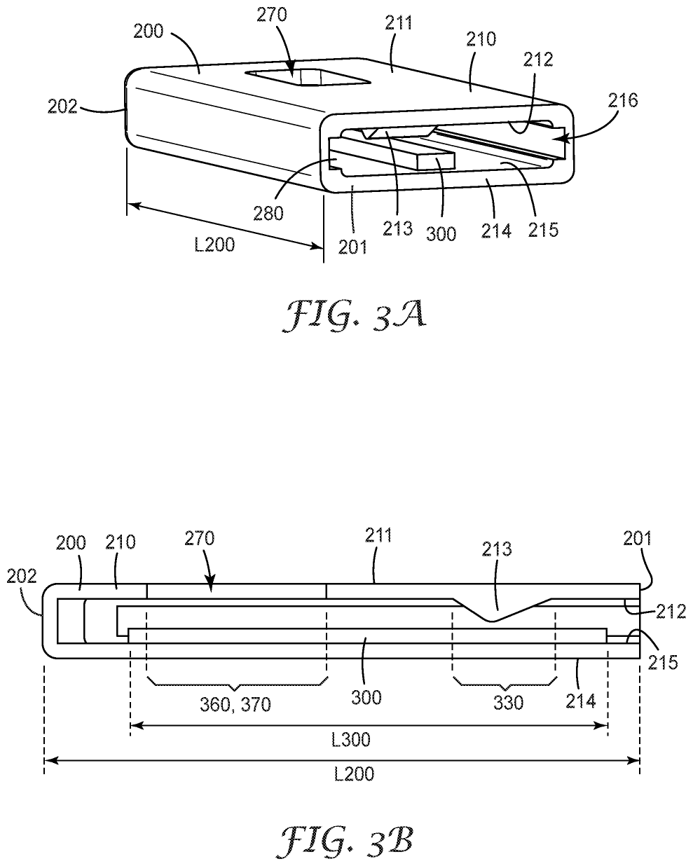

Referring now to FIGS. 1A-4B, a sample collection system 1 with a housing made up of slidably movable first and second parts 100, 200 are shown. The first part 100 contains porous sample collection media 130 and is constructed to slide inside the second part 200, which contains the assay 300.

The first part 100 has a body 110 defining a first end 101 and a second end 102, and a length L100 extending from the first end 101 to the second end 102. The body 110 has a first major surface 111, which in the drawings is shown as the upward-facing surface, and a second major surface 112 opposite to the first major surface 111. The body 110 has an opening 120 that extends through the body from the first major surface 111 to the second major surface. The opening 120 forms an airflow path through the first part 100.

An element of porous sample collection media 130 is disposed within the opening 120. The opening 120 may be occluded by the porous sample collection media 130. The porous sample collection media 130 may be attached or fixed to the body 110. The porous sample collection media 130 may be attached or fixed directly to the body 110 or may be fixed using a support element 124. In front of (upstream of) the porous sample collection media 130, the first part 100 may include a pre-filter or screen 132. The pre-filter or screen 132 may be provided as a layer of the porous sample collection media 130 on the upstream side. The pre-filter or screen 132 may be constructed to stay on the porous sample collection media 130 when the first part 100 is moved (e.g., slid) into the second part. Alternatively, the pre-filter or screen 132 may be constructed to come off (e.g., be peeled, curled, rolled back, crumble, etc.) of the porous sample collection media 130 when the first part 100 is moved (e.g., slid) into the second part.

The first part 100 may include an end piece 150 at the first end 101 of the body 110 to allow the first part 100 to be pulled out of and pushed into the second part 200. The first part 100, the second part 200, or both the first and second parts 100, 200 may include a stopping indicator that indicates to a user when to stop moving the first and second parts 100, 200 to register the porous sample collection media 130 with the sample receiving area 330 of the assay.

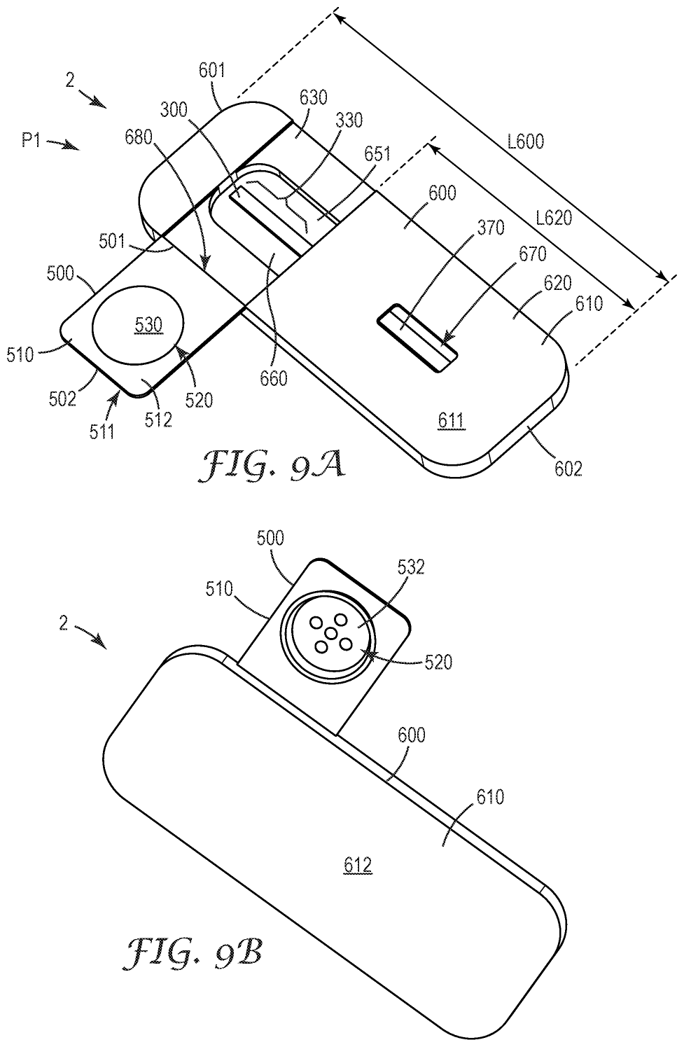

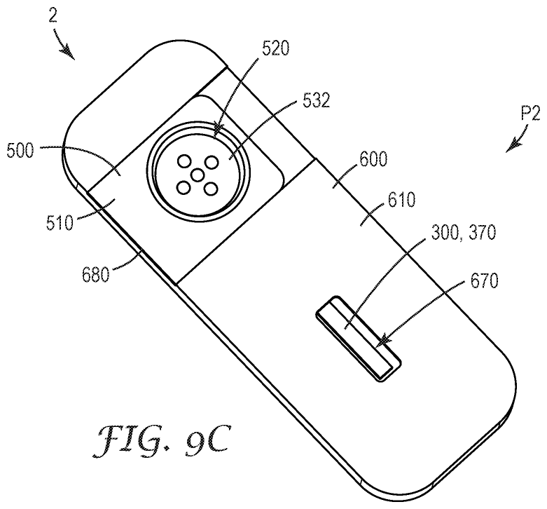

The second part 200 has a housing 210 with a first end 201 and a second end 202 and a length L200 extending from the first end 201 to the second end 202. The housing 210 has a first major wall 211 and a second major wall 214, defining an interior 216. The interior 216 may be a pocket constructed to receive the first part 100. The second part 200 may include an assay receptacle 260 or well (e.g., similar to assay receptacle 660 shown in FIG. 9A) for receiving an assay 300. The assay receptacle 260 may extend along the length L200 of the second part 200 from a sample receiving area 251 toward the second end 202. The sample receiving area 251 may be aligned with the opening 120 and the porous sample collection media 130 of the first part 100.

The assay 300 may be disposed in the assay receptacle 260, along an inside surface 215 of the second major wall 214. Any suitable assay may be used, such as a lateral flow assay or vertical flow assay. The assay 300 includes a sample receiving area 330 at or near the first end 301 of the assay and a test area 360 and result display 370 at or near the second end 302 of the assay. The assay 300 has a length L300 extending from the first end 301 to the second end 302. The length L300 of the assay 300 may be disposed parallel to the length L200 of the second part 200.

The second part 200 includes a result viewing area 270 that allows a user to view the result display 370 of the assay. The result viewing area 270 may be an opening in the first major wall 211 or may include a window. In some embodiments, the first major wall 211 or the entire housing 210 is made of a transparent material.

The second part 200 may include a protrusion 213 extending from an inside surface 212 of the first major wall 211. The protrusion 213 can be pressed down to cause more of the liquid 128 to be eluted from the porous sample collection media 130 onto the assay 300.

The second part 200 may include a track 280 to facilitate moving the first part 100 within the housing 210. The first part 100, the second part 200, or both the first and second parts 100, 200 may include a locking mechanism that locks the first and second parts 100, 200 in a closed position. The locking mechanism may include corresponding mating parts 190, 290 in the first and second parts 100, 200. The locking mechanism may irreversibly lock the first and second parts 100, 200 in the closed position such that the system 1 cannot be opened (that is, the first and second parts 100, 200 cannot be pulled apart) without breaking or distorting at least some of the parts.

The opening 120 in the first part 100 may include an extension 170 that allows the results of the assay 300 to be viewed when the first part 100 is pushed into the second part 200.

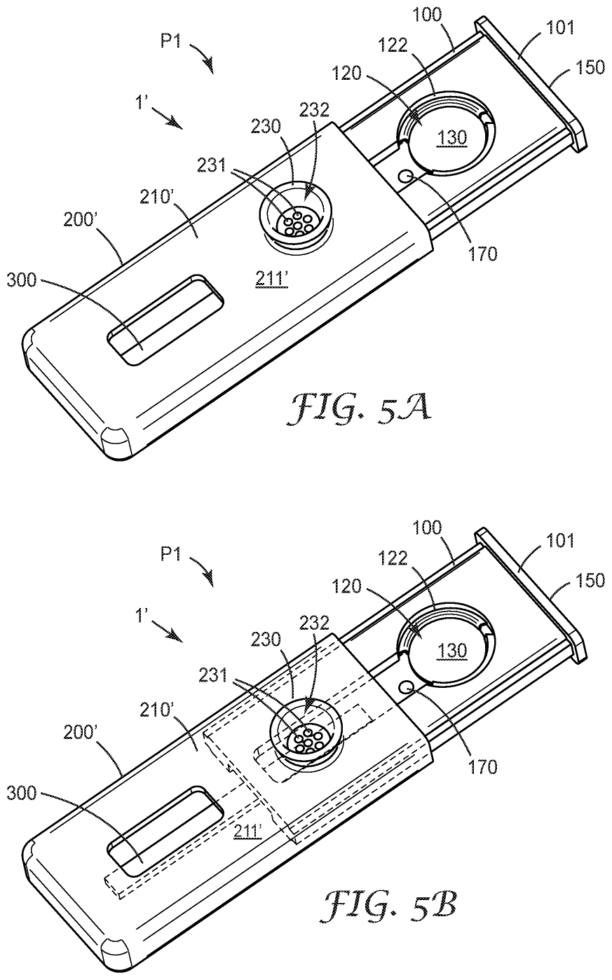

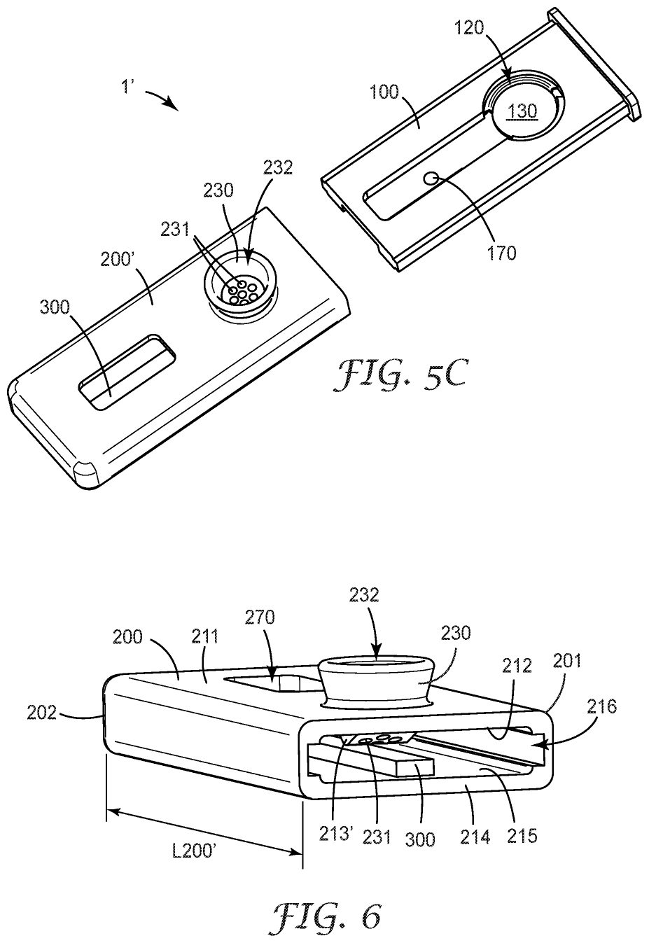

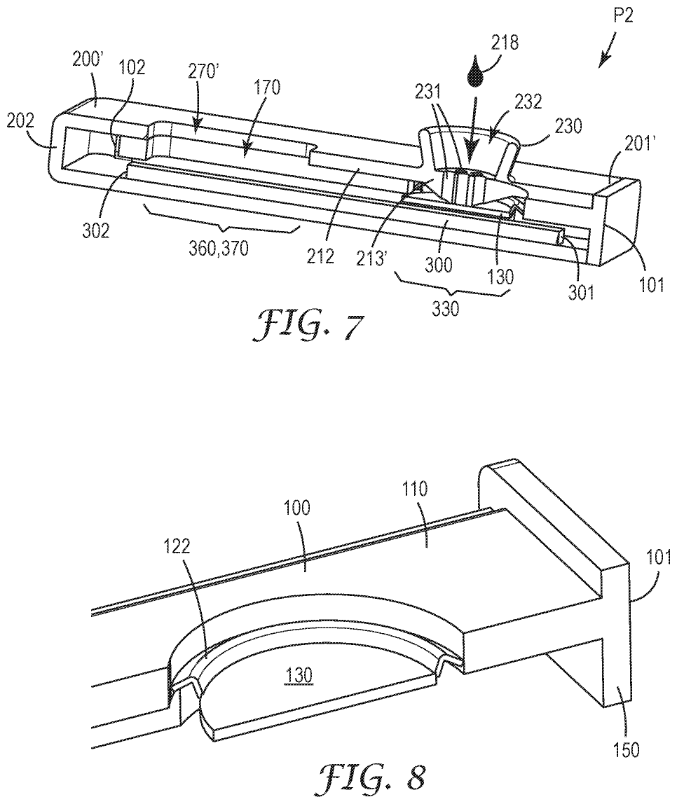

In some embodiments, as shown in FIGS. 5A-8, the second part 200′ includes a liquid inlet 230. The liquid inlet 230 may define one or more openings 231 extending through the first major wall 211′ of the second part 200. The liquid inlet 230 may further include a well 232. The well 232 may be provided to contain applied liquid 128 before the liquid 128 flows through the one or more openings 231 onto the porous sample collection media 130.

The second part 200′ may include a protrusion 213′ extending from an inside surface 212′ of the first major wall 211′. The protrusion 213′ can be pressed down to cause more of the liquid 128 to be eluted from the porous sample collection media 130 onto the assay 300. the openings 231 may extend through the protrusion 213′.

The first part 100′ may include features that support the porous sample collection media 130. The first part 100′ may include a circular flange 122 disposed in the opening 120. The porous sample collection media 130 may be seated in the circular flange 122.

In an alternative embodiment of the system 2, shown in FIGS. 9A-9C, the first part 500 is provided as a flap 510 connected to the second part 600 by a hinge 680. The flap 510 has a first side 501 and an opposing second side 502. The first side 501 is connected to the hinge 680. The flap 510 has a first major surface 511 and opposing second major surface 512 extending between the first side 501 and second side 502. The flap 510 includes an opening 520 extending through the flap from the first major surface 511 to the second major surface 512. The opening 520 provides the airflow path through the first part 500. The porous sample collection media 530 is disposed in and occludes the opening 520. Instead of one large opening 520, the flap 510 may include a plurality of small openings and the porous sample collection media 530 may be disposed against the plurality of openings, occluding the openings. In front of (upstream of) the porous sample collection media 530, the first part 500 may include a pre-filter or screen 532. The pre-filter or screen 532 may alternatively be provided as a layer of the porous sample collection media 530 on the upstream side.

The second part 600 has a housing 610 with a first end 601, a second end 602, and a length L600 extending from the first end 601 to the second end 602. The housing 610 includes a first major wall 611 and opposing second major wall 612 that form a pocket 620. The pocket 620 is constructed to house the assay 300. The pocket 620 has a length L620. The length L620 of the pocket 620 may be less than the overall length L600 of the housing, leaving an area above the pocket to accommodate the first part 100. This area may be slightly recessed, forming a flap receiving area 630. The housing 610 may further include an assay receptacle 660. A sample receiving area 651 may be disposed outside of the pocket 620, in the flap receiving area 630, such that when the flap 510 is closed, the porous sample collection media 530 overlays the sample receiving area 651.

The second part 600 includes a result viewing area 670 that allows a user to view the result display 370 of the assay. The result viewing area 670 may be an opening in the first major wall 611 or may include a window. In some embodiments, the first major wall 611 or the entire housing 610 is made of a transparent material.

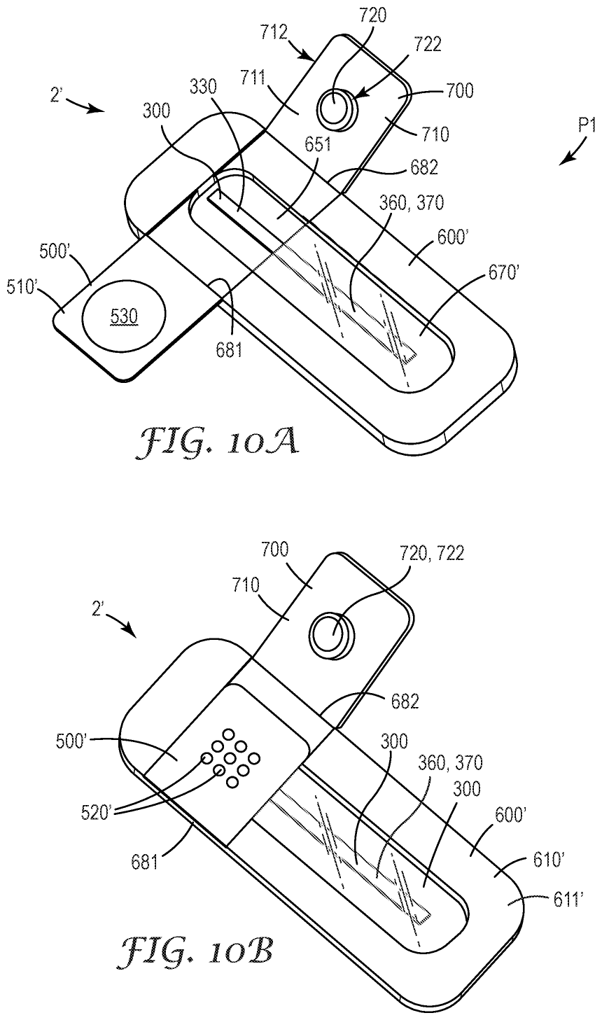

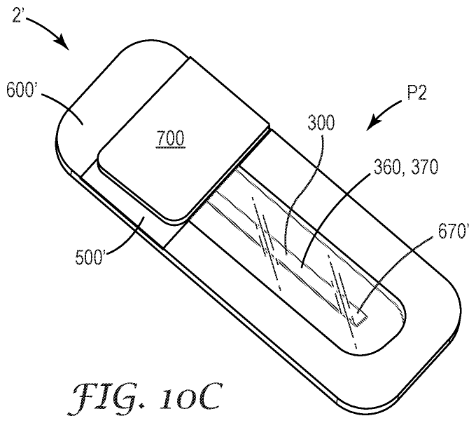

The system 2′ may further include a third part 700, shown in FIGS. 10A-10C, for providing a metered dose of liquid 722 for application onto the porous sample collection media 530. The third part 700 may be a flap 710, similar to the first part 500. The flap 710 is connected to the second part 600 by a second hinge 682. The second hinge 682 may be positioned along the side of the second part 600, opposite to the first hinge 681. According to an embodiment, the third part 700 may be closed over the first part 500 when the first part 500 is in a closed position, as shown in FIG. 10C.

The flap 710 of the third part 700 has a first major surface 711 that faces the second part 600 in a closed position, and a second major surface 712 opposite to the first major surface 711. The third part 700 includes a liquid reservoir 720. The liquid reservoir 720 may be provided on the first major surface 711. The liquid reservoir 720 contains the metered dose of liquid 722. The liquid reservoir 720 may be deformable and configured to discharges fluid from the metered fluid dose element upon a user applying pressure to the liquid reservoir 720. In some embodiments the liquid reservoir 720 is pierceable, frangible, or rupturable. A user may apply pressure to the liquid reservoir 720, for example, by first closing the first part 500 and the third part 700 and then pressing the third part 700 against the first part 500. The liquid reservoir 720 may include a deformable member 721, such as a pouch or layer made of foil or film or a glass ampule, that can easily be pierced or ruptured. In embodiments where the liquid reservoir 720 is pierceable, the first part 500 may include a piercing element or slicing element constructed to pierce the liquid reservoir 720. The pouch or ampule containing the metered dose of liquid 722 may be sterilized, for example by autoclaving or by using radiation, to sterilize the liquid. The metered dose of liquid 722 may remain sterile until it is used. The first part 500 may include a recess on the first major surface 511 for receiving the liquid from the liquid reservoir 720.

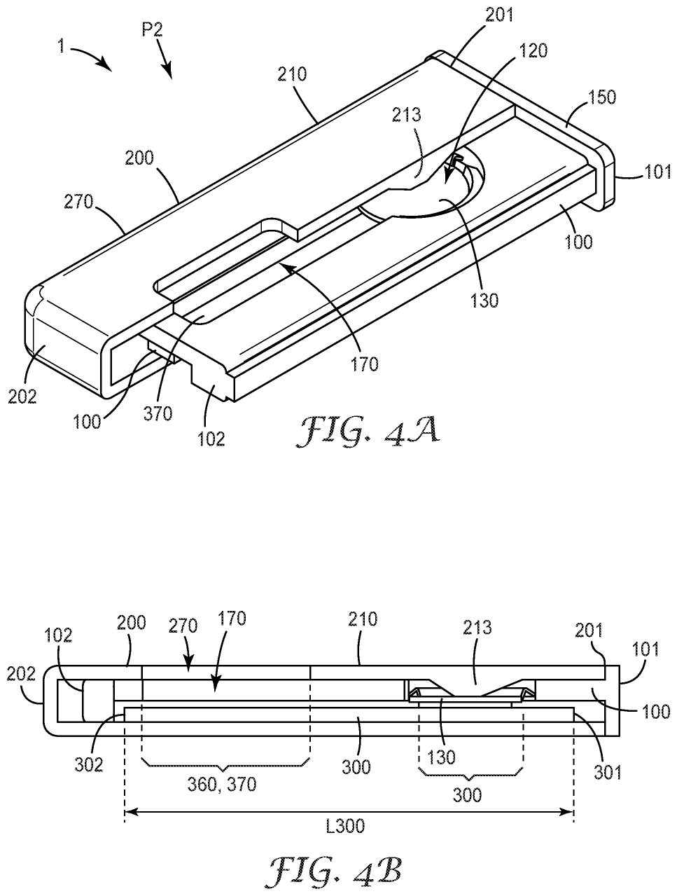

Although the first and second parts may be moved differently in the different embodiments, the general principle of using the systems 1, 2, 2′ in the various embodiments is the same. The first part 100, 500 and the second part 200, 600 have a first position P1 that is a sample collection position and a second position P2 that is a testing position. The first position P1 is shown, for example, in FIGS. 1A, 5A, 9A, and 10A, and the second position P2 is shown, for example, in FIGS. 4A, 8, 9C, and 10C. In the first position P1, the opening 120, 520 and the porous sample collection media 130, 530 of the first part 100, 500 are available for a user to inhale through the opening 120, 520. In the embodiments shown in FIGS. 1A-8, in the first position P1 the first part 100 is pulled out of (extends from) the second part 200. In the embodiments shown in FIGS. 9A-10C, in the first position P1 the flap 510 of the first part 500 is open. That is, the flap 510 extended away from the second part 600. In the second position P2, the porous sample collection media 130, 530 is aligned with the sample receiving area 330 of the assay 300. In the embodiments shown in FIGS. 1A-8, in the second position P2 the first part 100 is pushed into the second part 200. In the embodiments shown in FIGS. 9A-10C, in the second position P2 the flap 510 of the first part 500 is closed and overlaps the flap receiving area 630 of the second part 600.

In the first position P1, a user may exhale into the opening 120, 520. Exhalation airflow passes through the thickness of the screen 132, 532 (if included) and porous sample collection media 130, 530. The porous sample collection media 130 at least partially occludes the opening 120, 520. The porous sample collection media 130 may have a major plane that is orthogonal to the direction of the exhalation airflow passing through the thickness of the porous sample collection media 130. The porous sample collection media 130 captures viruses, pathogens, or other analytes, in the exhalation airflow that passes through the porous sample collection media 130.

In an alternative embodiment of the system 2, shown in FIGS. 9A-9C, the first part 500 is provided as a flap 510 connected to the second part 600 by a hinge 680. The flap 510 has a first side 501 and an opposing second side 502. The first side 501 is connected to the hinge 680. The flap 510 has a first major surface 511 and opposing second major surface 512 extending between the first side 501 and second side 502. However, instead of opening 520 and media 530, the flap 510 may house a small-sized swab that the user can remove from the flap 510 and use to obtain a sample (exhalation, nasal, throat, etc) from the user. This embodiment including a swab is not specifically shown in the drawings.

In the embodiment shown in FIGS. 1A-4B, the liquid is added to the porous sample collection media 130 before the first and second parts 100, 200 are moved to the second position P2.

In the embodiments shown in FIGS. 1A-8, the first and second parts 100, 200 may be moved to the second position P2 by pushing the first part 100 into the second part 200. The first part 100 may be pushed in until the porous sample collection media 130 overlaps the sample receiving area 330 of the assay. The first part 100 may be pushed in until it reaches the end of the second part 200. In some embodiments, the first and second parts 100, 200 include a locking mechanism. The first part 100 may be pushed in until the locking mechanism locks.

In the embodiments shown in FIGS. 9A-10C, the first and second parts 100, 200 may be moved to the second position P2 by closing the first part 100 over the second part 200 until the flap 510 overlaps the flap receiving area 630 of the second part 600.

In the second position P2 the sample can be eluted by applying a liquid 128 to the porous sample collection media 130, 530. The eluent passes through the porous sample collection media 130, 530 onto the sample receiving area 330 of the assay 300. The eluent travels to the test area 360 of the assay 300 by wicking or capillary action, where the possible target virus, target pathogen, or other target analyte from the sample react with a testing reagent. The result, indicating either the presence or absence of the target virus, target pathogen, or other target analyte of interest, is displayed in a result display 370. The result display 370 may be viewed through the result viewing area 270, 670 of the second part.

The sample collection system may further comprise a machine-readable optical label. Such labels may include, for example, a bar code and a QR (quick response) code. The machine-readable optical label may be configured to display the result of the assay. The machine-readable optical label may be used to read and record the result. An electronic reader capable of reading machine-readable optical labels may be used to read and record the result. An electronic reader may be, for example, a smart phone, a tablet, a laptop, or bar code reader or QR code reader. The electronic reader may further be used to transmit the result, for example, to a healthcare provider or to a database.

A method of using the sample collection system may include exhaling into the opening in the first part to capture a sample in the porous sample collection media; moving the first and second parts relative to one another to align the porous sample collection media with the assay (e.g., with the sample receiving area of the assay); applying a liquid to the porous sample collection media; and reading a result in the result display of the assay. In some cases, the liquid is applied to the porous sample collection media prior to aligning the porous sample collection media with the assay. The liquid may be applied in an amount suitable for eluting viruses, pathogens, or other analytes, captured in the porous sample collection media. A suitable amount of liquid may be determined as a ratio of liquid volume to the surface area of the porous sample collection media. For example, the volume of liquid may be in a range from 10 μm/cm2 to 400 μm/cm2, or from 10 μm/cm2 to 250 m/cm2, or from 50 μm/cm2 to 150 μm/cm2. In some embodiments, the volume of liquid is from 50 μm to 500 μm. The method may further include reading the result display of the assay using an electronic reader.

The sample collection system may be provided as a kit. The kit may include the sample collection system as discussed above, and instructions for collecting a sample and testing the sample using the assay. The instructions may include instructions to: exhale along the airflow path to capture a sample in the porous sample collection media; move the first and second parts relative to one another to align the porous sample collection media with the assay; apply a liquid to the porous sample collection media; and read a result in a result display of the assay. The instructions may further include instructions to read a result display of the assay using an electronic reader.

All references and publications cited herein are expressly incorporated herein by reference in their entirety into this disclosure, except to the extent they may directly contradict this disclosure. Although specific embodiments have been illustrated and described herein, it will be appreciated by those of ordinary skill in the art that a variety of alternate and/or equivalent implementations can be substituted for the specific embodiments shown and described without departing from the scope of the present disclosure. It should be understood that this disclosure is not intended to be unduly limited by the illustrative embodiments and examples set forth herein and that such examples and embodiments are presented by way of example only with the scope of the disclosure intended to be limited only by the claims set forth here.

1. A sample collection system comprising:
a housing comprising a first part and a second part, the first and second parts being movable relative to one another;
porous sample collection media disposed along an airflow path in the first part; and
an assay disposed on the second part and constructed to receive a sample captured by the porous sample collection media.

2. The sample collection system of claim 1, wherein the airflow path comprises a through opening in the first part.

3. The sample collection system of claim 1, wherein the first part comprises a screen disposed in the airflow path in front of the porous sample collection media.

4. The sample collection system of any one of claim 1, wherein the first and second parts are slidably movable relative to one another.

5. The sample collection system of any one of claim 1, wherein the first and second parts are rotatably movable relative to one another.

6. The sample collection system of claim 5, wherein the first and second parts are connected to one another by a hinge.

7. The sample collection system of any one of claim 1, wherein moving the first and second parts relative to one another aligns the porous sample collection media with at least a portion of the assay.

8. The sample collection system of any one of claim 1, wherein moving the first and second parts relative to one another causes the porous sample collection media to come into contact with the assay.

9. The sample collection system of claim 1, wherein the assay comprises a sample receiving area, and wherein moving the first and second parts relative to one another aligns the porous sample collection media with the sample receiving area.

10. The sample collection system of claim 1, wherein the assay comprises a sample receiving area, and wherein moving the first and second parts relative to one another causes the porous sample collection media to come into contact with the sample receiving area.

11. The sample collection system of claim 10, the housing comprising a protrusion constructed to apply pressure to the porous sample collection media to press the porous sample collection media against the assay.

12. The sample collection system of claim 11, the housing comprising a liquid inlet constructed to receive a liquid and to direct the liquid onto the porous sample collection media.

13. The sample collection system of claim 11, the housing comprising a liquid reservoir containing a metered dose of liquid.

14. The sample collection system of claim 13, wherein the metered dose of liquid has a volume of 50 μL to 500 μL.

15. The sample collection system of claim 14, wherein the metered dose of liquid comprises saline solution comprising a surfactant.

16. The sample collection system of claim 15, wherein the liquid reservoir is constructed to apply liquid onto the porous sample collection media.

17. The sample collection system of claim 13, wherein the liquid reservoir is pierceable, frangible, or rupturable.

18. The sample collection system of claim 13, wherein the liquid reservoir is disposed on a third part of the housing.

19. The sample collection system of claim 18, wherein the third part of the housing is movable relative to the first part, second part, or both first and second parts.

20. The sample collection system of claim 19, wherein the third part is connected to the first part or the second part by a hinge.

21-31. (canceled)
